(19) United States
(12) Patent Application Publication
(10) Pub. No.: US 2005/0142207 A1
(43) Pub. Date: Jun. 30, 2005

(54) Method for preparing polymeric microsphere by aqueous two phase emulsion process

(75) Inventors: Ae-June Wang, Hsinchu (TW); Yi-Fong Lin, Jhonghe City (TW); Chi-Heng Jian, Jiaosi Township (TW); Pei-Lin Wang, Chiayi City (TW); Shin-Jr Liu, Niaosong Township (TW)

Correspondence Address: THOMAS, KAYDEN, HORSTEMEYER & RISLEY, LLP, 100 GALLERIA PARKWAY, NW, STE 1750, ATLANTA, GA, 30339-5948, US

(73) Assignee: Industrial Technology Research Institute

(21) Appl. No.: 11/024,904

(22) Filed: Dec. 29, 2004

(30) Foreign Application Priority Data
Dec. 31, 2003 (TW) 92137631

(51) Int. Cl. A61K 9/14; A61K 9/16; A61K 9/50

(52) U.S. Cl. 424/490; 264/4.1

(57) ABSTRACT

A method for preparing polymeric microsphere by an aqueous two phase emulsion process. A first polymer aqueous solution is provided and the first polymer includes a functional group capable of forming cross-linking. A second polymer aqueous solution is provided, which is acidic and miscible with the first polymer aqueous solution. The first and second polymer aqueous solutions are mixed and stirred to form an emulsion, such that the first polymer solution forms a dispersed phase in a continuous phase of the second polymer solution. The dispersed phase includes a plurality of the first polymeric microsphere, and a solidification film formed by cross-linking of the functional group constitutes a microsphere surface. Finally, the first polymeric microsphere are separated out.

### BACKGROUND OF THE INVENTION

1. Field of the Invention

The present invention relates to a method for preparing polymeric microspheres by an aqueous two phase emulsion process, and more particularly to a method for preparing polymeric microspheres by a aqueous two phase emulsion process using two miscible polymer solutions.

2. Description of the Related Art

Polymeric microsphere preparation methods can be classified into both spraying and emulsion methods. The spraying method can be seen in U.S. Pat. No. 6,238,705. A polymer with cross-linking properties, such as sodium alginate, is sprayed from a nozzl into an ionic cross-linking agent with a +2 charge and then Chitosan is adsorbed on the surface of the microspheres. This method does-not use an organic solvent or a surfactant in the process. However, the recovery yield is poor and is approximately 20-30% due to the nozzle spray wastage.

Regarding to the emulsion method, an oil/water emulsion method for preparing such polymeric microspheres is first provided, such as an oil-in-water or water-in-oil emulsion method. For example, in EP-0480729, a lipophilic drug, such as steroid drug or anticancer drug, is dissolved in an oil phase and then emulsified into an aqueous phase (a polysaccharide polymer or a mixture of many polysaccharide polymers), thus forming oil-in-water polymeric microsphere. The drawbacks of the above oil/water emulsion method suffers from the usage of an organic solvent or surfactant. Also, high temperature must be used to remove the organic solvent during the preparation process. Since the biological drugs such as peptide and protein are less stable than the small molecule drugs. Use of organic solvents or surfactants will denature the biological drugs and lose their activities.

In order to avoid the need for an organic solvent and a surfactant while achieving the requirement of high recovery, an aqueous two phase method has been applied to prepare polymeric microspheres. In 1995, Gehrke et al. provide a dextran/PEG aqueous two phase system. This system composed of two immiscible soluble polymers (Proceed. Intern. Symp. Control Rel. Bioact. Material., 22, 145-146).

EP 0213303 discloses many aqueous-two-phase systems and the polymer compositions are dextran-alginate/PEG, carboxymethylcellulose/PEG, and starch/PEG. The same, each system is composed of two immiscible polymers.

In U.S. Pat. No. 5,204,108, Illum et al. uses an aqueous two phase system, such as starch/PEG, albumin/PEG, or gelatin/PEG, to encapsulate insulin. Still, this system is composed of two immiscible polymers. In addition, glutaldehyde is used as a microsphere cross-linking agent.

Lamberti et al. in U.S. Pat. No. 5,827,707 provides a dextran-alginate/PEG system, which includes two immiscible polymers. Alginate is cross-linked to prepare as an implantable microcapsule.

In 2001, Hennink et al. in U.S. Pat. No. 6,303,148 disclose a controlled release aqueous two phase system, such as dextran-GMA/PEG and dextran-lactHEMA/PEG. The modified dextran-GMA can be cross-linked to form microspheres without the need of alginate. This system can be used to encapsulate protein drug or gene wherein at least 80 wt % of the microspheres had a particle size between 100 nm and 1000 μm.

From the above literatures and patents for the preparation of polymeric microspheres, the spraying method has poor recovery yield, and the oil/water emulsion method easily denature the encapsulated biological drug during the process. The Aqueous-two-phase method must use two immiscible polymers, which limits the polymer selections.

### SUMMARY OF THE INVENTION

An object of the present invention is to solve the above-mentioned problems and provide an aqueous two phase emulsion method for preparing polymeric microspheres. The present invention does not require any organic solvents or surfactants. Therefore, the encapsulated biological drug will not deactivated during the preparation process. Also, recovery yield and encapsulation efficiency of the drug will be increased.

To achieve the above objects, the aqueous two phase emulsion method for preparing polymeric microspheres includes the following steps. A first polymer aqueous solution is provided and the first polymer includes a functional group capable of forming cross-linking property. A second polymer aqueous solution is provided, which is acidic and miscible with the first polymer. The first and second polymer aqueous solutions are mixed and stirred to form an emulsion, such that the first polymer solution forms a dispersed phase in a continuous phase of the second polymer solution. The dispersed phase includes a plurality of the first polymeric microsphere, and a solidification film formed by the cross-linking functional group constitutes a microsphere surface. Finally, the first polymeric microsphere are separated from the solution.

The polymeric microspheres prepared by the aqueous two phase process can be used to encapsulate a drug. Therefore, the present invention also provides a method for preparing polymeric microspheres encapsulated with a drug, which includes the following steps. A first polymer aqueous solution is provided and the first polymer includes a functional group capable of forming cross-linking properties. A second polymer aqueous solution is provided, which is acidic and miscible with the first polymer aqueous solution. A drug and the first polymer aqueous solution are mixed to form a drug aqueous solution. The drug aqueous solution and the second polymer aqueous solution are mixed and stirred to form an emulsion, such that the first polymer aqueous solution forms a dispersed phase in a continuous phase of the second polymer aqueous solution. The dispersed phase includes a plurality of the first polymeric microsphere encapsulated with the drug, having a microsphere surface composed of a solidification film formed by cross-linking of the functional group. Finally, the first polymeric microsphere are separated out.

### BRIEF DESCRIPTION OF THE DRAWINGS

The present invention will be fully understood from the detailed description given herein below and the drawings, given by way of illustration only and thus not intended to be limitative of the present invention.

FIG. 1 shows the surface cross-linking theory of the polymer by hydrogen bonding.

FIG. 2 shows ionic cross-linking property of the polymer.

FIG. 3 is a schematic diagram shows the aqueous two phase process for preparing polymeric microsphere in continuous homogenization.

### DETAILED DESCRIPTION OF THE INVENTION

The aqueous two phase emulsion process of the present invention uses two miscible polymer solutions. One polymer (the first polymer) has a functional group capable of forming surface cross-linking property. For example, the first polymer can be a carboxylate polymer, that is, a polymer with a carboxylate (COO⁻) or carboxyl (COOH) group. Representative examples include alginic acid, alginate, propylene glycol alginate, carboxylmethyl cellulose, polyacrylic acid, and polyacrylate derivatives.

The other polymer (the second polymer) is not limited, provided that it is miscible with the first polymer. Representative examples of the second polymer include chitosan, starch, dextran, hydroxyl propyl methyl cellulose, and gelatin.

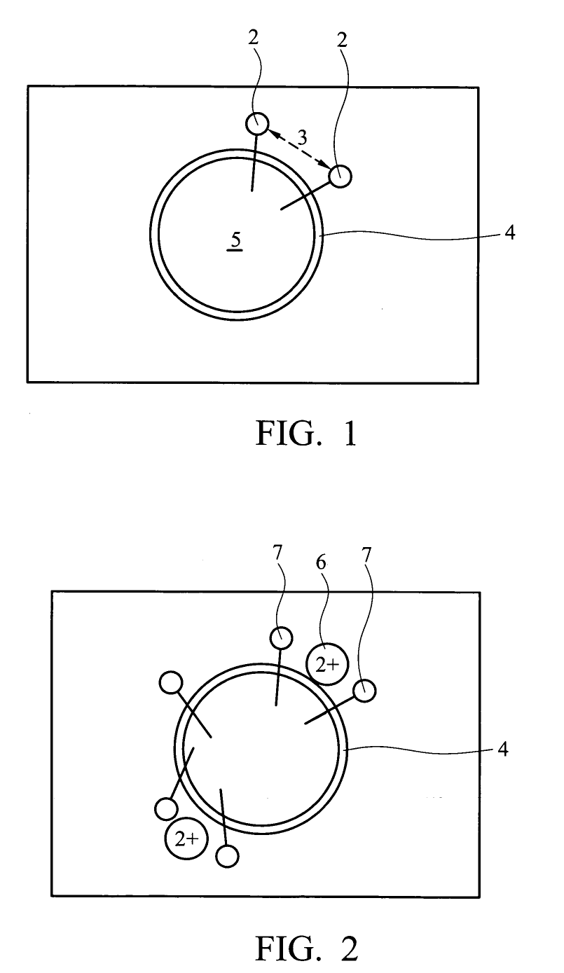

The second polymer solution 1 is adjusted to its pH to acidic. Then, the first and the second polymer aqueous solutions are mixed and stirred, for example, homogenized in a homogenizer, to form an emulsion. The first polymer aqueous solution forms a dispersed phase (including a plurality of microspheres) in a continuous phase of the second polymer aqueous solution. Since the COO⁻ or COOH groups 2 in the first polymer form hydrogen bonds 3 between each other and are cross-linked, a solidification film 4 will form on the surface of each microsphere of the first polymer 5 (carboxylate polymer) as shown in FIG. 1. The solidification film (protective film) by surface cross-linking can prevent mutual dissolution of the inner and outer polymers.

Subsequently, in order to stabilize and enhance the polymeric microsphere strength, a cross-linking agent 6, such as an ionic cross-linking agent with +2 valence, can be added to initiate cross-linking between COO⁻7 and the ionic cross-linking agent as shown in FIG. 2. The polymeric microsphere obtained from the present invention has a particle size between 0.1 μm and 100 μm.

According to the present invention, the second polymer is preferably adjusted to acidity, for example, to pH 0.5 to 6, most preferably pH 1.5 to 5. Generally, the cross-linking agent added has almost the same pH as the second polymer aqueous solution. The pH of the first polymer aqueous solution is not limited and can, for example, be 2 to 13.

The first polymer aqueous solution can have a concentration higher than 1%, preferably 2% to 10%. The second polymer aqueous solution can have a concentration higher than 0.5%, preferably 1% to 10%.

The weight of the second polymer aqueous solution can be 1.5 to 20 times, preferably 2 to 3 times, the weight of the first polymer aqueous solution.

The polymer obtained from the present invention, prepared by the aqueous two phase emulsion process using two miscible polymer solutions, can be used to encapsulate a drug. The process is described below. A drug and the first polymer aqueous solution are mixed to form a drug aqueous solution. The second polymer aqueous solution is adjusted to acidity. Then, the drug aqueous solution and the second polymer aqueous solution are mixed and stirred, for example, homogenized in a homogenizer, to form an emulsion. The first polymer aqueous solution forms a dispersed phase (including a plurality of the first polymeric microsphere encapsulated with the drug) in a continuous phase of the second polymer aqueous solution.

As mentioned above, since the COO⁻ or COOH groups in the first polymer form hydrogen bonds between each other and are cross-linked, a solidification film will form on the surface of each microsphere of the first polymer as shown in FIG. 1. The drug is encapsulated in the microsphere but not shown. The solidification film (protective film) by surface cross-linking prevents mutual dissolution of the inner and outer polymers. Also, release of the drug to the outer phase is prevented by the solidification film, thus increasing encapsulation efficiency (E.E.)

Subsequently, in order to stabilize and strengthen the polymeric microsphere, a cross-linking agent, such as an ionic cross-linking agent with +2 valence, can be added to initiate cross-linking between COO⁻ and the ionic cross-linking agent as shown in FIG. 2. The drug-encapsulated polymeric microsphere obtained from the present invention has a particle size between 0.1 μm and 100 μm.

According to the present invention, drugs suitable for encapsulation in the polymer microsphere are not limited, for example, peptide, protein or liposomes of various electricity.

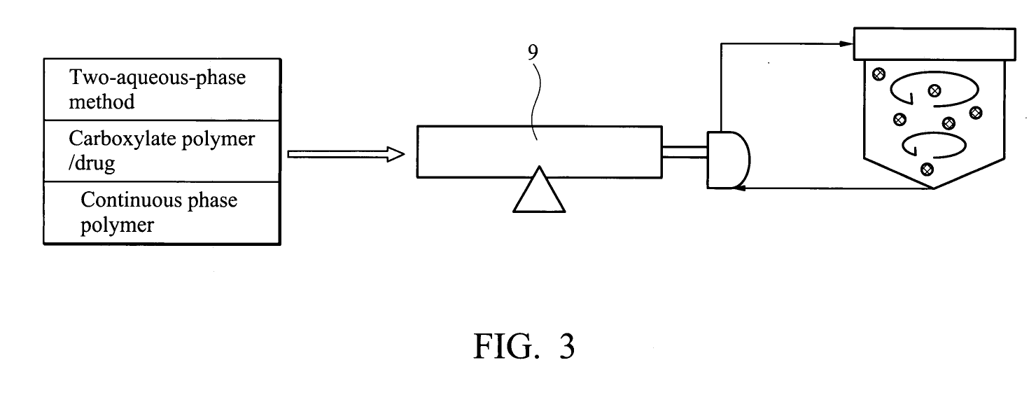

Homogenization used in the present invention can be batch homogenization or continuous homogenization. The method of the present invention is suitable for scale up process. After the aqueous-two-phase emulsion process is performed, continuous homogenization 9 (shown in FIG. 3) is preferably used for the large scale emulsion.

The following examples are intended to illustrate the process and the advantages of the present invention without limiting its scope, since numerous modifications and variations will be apparent to those skilled in the art.

### EXAMPLE 1

Preparation of Polymeric Microspheres

1 g of sodium alginate was completely dissolved to form a 10% sodium alginate aqueous solution. 2 g of chitosan was dissolved to form a 1.5% aqueous solution (pH 4.4). These two aqueous solutions were mixed and homogenized at 9500 rpm for 30 minutes to form an emulsion. 1 g of calcium chloride solution (4.5%, pH 4.4) was added dropwise to the emulsion and stirred for 30 minutes, allowing sodium alginate to crosslink to form polymeric microsphere. The resulting microspheres were filtered off under reduced pressure. The filter cake was dispersed into water for 10 minutes (filter cake:water=1:3(w/w)), and then frozen at −20° C. for 3 hours. After complete freezing, the sample was freeze-dried for 24 hours, that is, frozen at −40° C. for 60 minutes and then dried at 4° C. until completely dry, obtaining dried polymeric microspheres.

### EXAMPLE 2

Preparation of Polymeric Microspheres

1 g of sodium alginate was completely dissolved to form a 10% sodium alginate aqueous solution. 2 g of dextran was dissolved to form a 10% aqueous solution (pH 1.0). These two aqueous solutions were mixed and homogenized at 9500 rpm for 30 minutes to form an emulsion. 1 g of calcium chloride solution (6%, pH 1.0) was added dropwise to the emulsion and stirred for −30 minutes, allowing sodium alginate to crosslink to form polymeric microspheres. The resultant microspheres were filtered off under reduced pressure. The filter cake was dispersed in water for 10 minutes (filter cake:water=1:3(w/w)), and then frozen at −20° C. for 3 hours. After complete freezing, the sample was freeze-dried for 24 hours, that is, frozen at −40° C. for 60 minutes and then dried at 4° C. until completely dry, obtaining dried polymeric microsphere.

### EXAMPLE 3

Preparation of Polymeric Microspheres

1 g of Carbopol 934P (CP 934P, manufactured from BF Goodrich) was completely dissolved in 0.5 N NaOH to form a 3% Carbopol aqueous solution (pH 13). 2 g of chitosan was dissolved in water to form a 2% aqueous solution (pH 2.0). These two aqueous solutions were mixed and homogenized at 9500 rpm for 30 minutes to form an emulsion. 1 g of zinc sulfate solution (6%, pH 2.0) was added dropwise to the emulsion and stirred for 30 minutes, allowing Carbopol to crosslink to form polymeric microspheres. The resultant microspheres were filtered off under reduced pressure. The filter cake was dispersed in water for 10 minutes (filter cake:water=1:3(w/w)), and then frozen at −20° C. for 3 hours. After complete freezing, the sample was freeze-dried for 24 hours, that is, frozen at −40° C. for 60 minutes and then dried at 4° C. until completely dry, obtaining dried polymeric microspheres.

### EXAMPLE 4

Preparation of Polymeric Microspheres encapsulated with calcitonin liposomes 1 g of sodium alginate was completely dissolved in water to form a 10% sodium alginate aqueous solution, and then mixed with the same amount of a calcitonin liposomes solution. After complete dissolution, 1 g of the sodium alginate/liposomes solution and 2 g of a chitosan solution (1.5%, pH 4.4) were mixed and homogenized at 9500 rpm for 30 minutes to form an emulsion. 1 g of calcium chloride solution (4.5%, pH 4.4) was added dropwise to the emulsion and stirred for 30 minutes, allowing sodium alginate to crosslink to form calcitonin liposomes polymeric microsphere (encapsulation efficiency (E.E.) was higher than 70.7%). The resultant microspheres were filtered off under reduced pressure. The filter cake was dispersed in water for 10 minutes (filter cake:water=1:3(w/w)), and then frozen at −20° C. for 3 hours. After complete freezing, the sample was freeze-dried for 24 hours, that is, frozen at −40° C. for 60 minutes and then dried at 4° C. until completely dry, obtaining dried polymeric microspheres.

### EXAMPLES 5-19

The same procedures as described in Example 4 were employed, except that some conditions were changed. The differentiating conditions and results are shown in Table

TABLE 1

| | Calcitonin | Sodium | | | | | |
|---|---|---|---|---|---|---|---|
| | Liposomes | Alginate | Chitosan | Chitosan | | | |
| | conc. | conc. | 72 KDa | 180 KDa | CaCl₂ | | E.E. |
| Example | (mg/mL) | (%) | (%) | (%) | (%) | ZnSO_{4 (%)} | (%) |
| 5 | 0.25 | 5 | 1.5, | | 4.5 | | 90.0 |
| | | | pH 2.0 | | pH 2.0 | | |
| 6 | 0.5 | 5 | 1.5, | | | 4.5 | 93.8 |
| | | | pH 2.0 | | | pH 2.0 | |
| 7 | 0.67 | 3.3 | 1.5, | | 4.5 | | 71.0 |
| | | | pH 2.0 | | pH 2.0 | | |
| 8 | 0.67 | 3.3 | 2, | | 4.5 | | 84.9 |
| | | | pH 2.0 | | pH 2.0 | | |
| 9 | 0.33 | 3.3 | 2, | | 6 | | 74.1 |
| | | | pH 2.0 | | pH 2.0 | | |
| 10 | 0.33 | 3.3 | 2, | | 6 | | 83.2 |
| | | | pH 2.0 | | pH 2.0 | | |
| 11 | 0.33 | 3.3 | 2, | | | 6 | 94.5 |
| | | | pH 2.0 | | | pH 2.0 | |
| 12 | 0.33 | 3.3 | | | | 6 | 88.5 |
| | | | pH 2.0 | 1 | | pH 2.0 | |
| 13 | 0.37 | 2.5 | 2 | | | 6 | 59.9 |
| | | | pH 2.0 | | | pH 2.0 | |
| 14 | 0.37 | 2.5 | 1 | | | 6 | 55.5 |
| | | | pH 2.0 | | | pH 2.0 | |
| 15 | 0.4 | 2 | 2 | | | 6 | 62.0 |
| | | | pH 2.0 | | | pH 2.0 | |
| 16 | 0.4 | 2 | 1 | | | 6 | 59.8 |
| | | | pH 2.0 | | | pH 2.0 | |
| 17 | 0.37 | 2.5 | | 2 | | 6 | 91.8 |
| | | | | pH 2.0 | | pH 2.0 | |
| 18 | 0.37 | 2.5 | | 1 | | 6 | 89.8 |
| | | | | pH 2.0 | | pH 2.0 | |
| 19 | 0.4 | 2 | | 2 | | 6 | 65.9 |
| | | | | pH 2.0 | | pH 2.0 | |

### EXAMPLE 20

Preparation of Polymeric Microsphere Encapsulated with Insulin Liposome

10% sodium alginate solution, 1.5% chitosan solution, and 4.5% of calcium chloride solution were prepared and adjusted to pH 2.0. 0.33 mL of an insulin liposome solution and 0.67 g of 10% the sodium alginate solution were mixed and then added to 2 mL of the chitosan solution. The mixed solution was homogenized at 9500 rpm for 1 minute to form an emulsion. 1 mL of 4.5% calcium chloride solution was added dropwise to the emulsion and stirred for 5 minutes, obtaining a polymer microsphere solution encapsulated with insulin. The resultant microspheres were filtered off under reduced pressure. The filter cake was dispersed in water for 10 minutes (filter cake:water=1:3(w/w)), and then frozen at −20° C. for 3 hours. After complete freezing, the sample was freeze-dried for 24 hours, that is, frozen at −40° C. for 60 minutes and then dried at 4° C. until completely dry, obtaining dried polymeric microsphere.

### EXAMPLES 21-29

The same procedures as described in Example 20 were employed, except that some conditions were changed. The differentiating conditions and results are shown in Table 2.

TABLE 2

| | Insulin | | | | | |
|---|---|---|---|---|---|---|
| | Liposome | Sodium | | | | |
| Exam- | conc. | Alginate | CP 934P | Chitosan | CaCl₂ | E.E. |
| ple | (mg/mL) | (%) | (%) | (%) | (%) | (%) |
| 21 | 4.0 | 3.3 | | 1.5 | 4.5 | 88.9 |
| | | | | pH 2 | | |
| 22 | 4.0 | 3.3 | | 1.0 | 6 | 30.7 |
| | | | | pH 1 | pH 1 | |
| 23 | 4.0 | 3.3 | | 1.0 | 6 | 94.1 |
| | | | | pH 2 | pH 2 | |
| 24 | 4.0 | 3.3 | | 1.0 | 6 | 77.9 |
| | | | | pH 3 | pH 3 | |
| 25 | 4.0 | 3.3 | | 1.0 | 6 | 74.3 |
| | | | | pH 4 | pH 4 | |
| 26 | 4.0 | 3.3 | | 1.0 | 6 | 97.4 |
| | | | | pH 5 | pH 5 | |
| 27 | 4.0 | 3.3 | | 1.0 | 6 | 97.4 |
| | | | | pH 5.85 | pH 5.85 | |
| 28 | 4.0 | 1.7 | 1.7 | 1.5 | 4.5 | 87.6 |
| | | | | pH 2 | | |
| 29 | 4.0 | 1.1 | 2.2 | 1.5 | 4.5 | 97.1 |
| | | | | pH 2 | | |

### Example 30

Preparation of Polymeric Microspheres by Aqueous Two Phase Method in Six Repetitions

The same procedures as described in Example 20 were employed, except that the concentration of sodium alginate was changed. Six repetitions were performed. The results are shown in Table 3.

TABLE 3

| | Sodium | | | | Drug amount |
|---|---|---|---|---|---|
| | Alginate | Chitosan | CaCl₂ | E.E. | (mg/g |
| Example | (%) | (%) | (%) | (%) | micropheres) |
| 30-1 | 3.3 | 1.5 pH 2 | 4.5 pH 2 | 88.9 | * |
| 30-2 | 3.3 | 1.5 pH 2 | 4.5 pH 2 | 84.1 | * |
| 30-3 | 3.3 | 1.5 pH 2 | 4.5 pH 2 | 87.3 | * |
| 30-4 | 3.3 | 1.5 pH 2 | 4.5 pH 2 | 82.5 | 39.7 |
| 30-5 | 3.3 | 1.5 pH 2 | 4.5 pH 2 | 86.9 | 37.6 |
| 30-6 | 3.3 | 1.5 pH 2 | 4.5 pH 2 | 87.4 | 38.2 |

It can be seen from Table 3 that the aqueous two phase method for preparing sodium alginate polymeric microspheres exhibits good repeatability. In addition, the encapsulation efficiency (E.E.) of insulin liposome is as high as 86.2%, and the CV (coefficient of variation) (%)=2.77%.

### Comparative Examples 31 and 32

The same procedures as described in Example 21 were employed, except that the spray nozzle method was used instead. The obtained polymeric microspheres encapsulated with insulin liposomes was 0.1 g. Table 4 shows a comparison between the results of Example 21, Comparative Example 31 and 32.

TABLE 4

| | | Particle | | | | | |
|---|---|---|---|---|---|---|---|
| | | Size of | | Drug | | | |
| | Polymer | Polymer | | Content | | | |
| | Micropheres | Micropheres | E.E. | (mg/g | Recovery | | |
| Example | (g) | (μm) | (%) | microphere) | (%) | Method | Apparatus |
| Comp. | 0.1 | 27.37 | 93.7 | 20.7 | 76.4 | Spray | 0.54 mm |
| Exp. 31 | | | | | | Nozzle | Nozzle |
| Comp. | 0.1 | 15.08 | 85.4 | 21.3 | 76.4 | Spray | 0.54 mm |
| Exp. 32 | | | | | | Nozzle | Nozzle |
| Example | 0.1 | 2.51 | 88.9 | 37.8 | 90.1 | Two-aqueous- | Probe type |
| 21 | | | | | | phase | homogenizer |
| | | | | | | Emulsion | |

It can be seen from Table 4 that the aqueous two phase method of the present invention provides polymeric microsphere with a high recovery of 90%. However, using the conventional spray nozzle method to prepare polymeric microsphere only obtains a recovery yield of 74-76%

### EXAMPLE 33

The same procedures as described in Example 21 were employed, except that insulin liposome was not encapsulated and the reactant amounts were scaled up such that the obtained sodium alginate polymeric microsphere was 5 g. Table 5 shows the result of triplet repetitions.

TABLE 5

| | | Particle | | | |
|---|---|---|---|---|---|
| | | Size of | Rate of | Homog- | Cross- |
| | Polymer | Polymer | Homog- | enization | linking |
| | Microspheres | Micropheres | enizer | Time | Time |
| Example | (g) | (μm) | (rpm) | (min) | (min) |
| 33-1 | 5 | 2.09 | 3000 | 1 | 5 |
| 33-2 | 5 | 2.09 | 5000 | 1 | 5 |
| 33-3 | 5 | 2.12 | 3000 | 5 | 5 |

This example enlarges the aqueous two phase process to prepare 5 g of polymeric microsphere using a homogenizer at 3000-5000 rpm for 1-5 minutes. The obtained sodium alginate polymeric microsphere had a relative uniform particle size, the average particle size was 2.10 μm, and CV (%)=0.85%.

### EXAMPLE 34

400 g, 10% sodium alginate solution, 2000 mL, 1.5% chitosan solution, and 1000 mL, 4.5% of calcium chloride solution were prepared. Then, the chitosan and calcium chloride solutions were adjusted to pH 2. 400 g of 10% sodium alginate solution and 800 g of an insulin liposome solution were mixed to form 1200 g of a mixed solution.

After complete mixing, 1000 g of the sodium alginate/insulin liposome solution was added to 2000 mL of chitosan solution and then homogenized by a continuous homogenizer and a 5 Liter circulation tube at 21000 rpm for 60 minutes to form an emulsion. 1000 mL of the calcium chloride solution was then added slowly and the mixture stirred at 250 rpm for 30 minutes in order to cross-link sodium alginate to form insulin liposome microspheres. The reaction solution was poured in a 4 Liter plate-type filter press in two batches and filter pressed at 3 kg/cm² for separation. The obtained filter cake was dispersed in water (filter cake:water=1:3(w/w)), then poured in a 35 cm×25cm stainless steel plate (the liquid height was not higher than 0.5 cm), and then frozen at −20° C. for 3 hours. After complete freezing, the sample was freeze-dried for 24 hours, that is, frozen at −40° C. for 60 minutes and then dried at 4° C. until completely dry, obtaining 100 g of dried insulin polymeric microspher. The encapsulation efficiency reached up to 87.8% and the recovery reached up to 94.8%.

### EXAMPLES 35-37

The same procedures as described in Example 34 were employed, except that the reactant amounts were changed such that the obtaining insulin liposome-encapsulated sodium alginate microspheres amounts were different. The results are shown in Table 6.

### EXAMPLES 38 and 39

The same procedures as described in Example 34 were employed, except that the reactant amounts were changed such that the obtaining insulin liposome-encapsulated sodium alginate microspheres amounts were different, and that a continuous homogenization method was used. The results are shown in Table 6.

TABLE 6

| | | Particle | | | | | |
|---|---|---|---|---|---|---|---|
| | | Size of | | Drug | | | |
| | Polymer | Polymer | | Content | | | |
| | Micropheres | Micropheres | E.E. | (mg/g | Recovery | | |
| Example | (g) | (μm) | (%) | microphere) | (%) | Method | Apparatus |
| 35 | 0.1 | 2.51 | 88.9 | 37.8 | 90.7 | Batch | Probe type |
| | | | | | | | homogenizer |
| 36 | 5 | 2.59 | 90.1 | 39.4 | 91.3 | Batch | Probe type |
| | | | | | | | Homogenizer |
| 37 | 10 | 2.49 | 88.5 | 38.9 | 89.4 | Batch | Probe type |
| | | | | | | | Homogenizer |
| 38 | 50 | 2.29 | 90.2 | 38.5 | 94.0 | Continuous | Continuous |
| | | | | | | | type |
| | | | | | | | Homogenizer |
| 39 | 100 | 3.27 | 89.4 | 38.0 | 94.7 | Continuous | Continuous |
| | | | | | | | type |
| | | | | | | | Homogenizer |

It can be seen from Table 6 that using the aqueous two phase method of the present invention obtains 4 liters of emulsion and 100 g of dried polymeric microsphere. Also, the polymeric microsphere had good encapsulation efficiency and the drug content had good repeatability. In addition, when the process was changed to continuous, the recovery was increased from 90% to 94%.

In conclusion, the present invention uses two miscible polymer solutions to perform emulsion, adjusts the continuous polymer solution to acidity, and allows the dispersed phase to have surface cross-linking to form a solidification film, obtaining polymeric microsphere. The present invention does not require any organic solvent or surfactant. Therefore, the encapsulated biological drug is not deactivated. The recovery and the encapsulation efficiency of the drug are high.

The foregoing description of the preferred embodiments of this invention has been presented for purposes of illustration and description. Obvious modifications or variations are possible in light of the above teaching. The embodiments chosen and described provide an excellent illustration of the principles of this invention and its practical application to thereby enable those skilled in the art to utilize the invention in various embodiments and with various modifications as are suited to the particular use contemplated. All such modifications and variations are within the scope of the present invention as determined by the appended claims when interpreted in accordance with the breadth to which they are fairly, legally, and equitably entitled.

1. A method for preparing polymeric microspheres by an aqueous-two-phase emulsion process, comprising the following steps:
providing an aqueous solution of a first polymer, the first polymer having a functional group capable of forming surface cross-linking;
providing an acidic aqueous solution of a second polymer, wherein the first and second polymer aqueous solutions are miscible;
mixing the first and second polymer aqueous solutions and stirring to form an emulsion, such that the first polymer aqueous solution forms a dispersed phase in a continuous phase of the second polymer aqueous solution, wherein the dispersed phase includes a plurality of the first polymeric microsphere having a microsphere surface composed of a solidification film formed by cross-linking of the functional group; and separating the first polymeric microsphere out.

2. The method as claimed in claim 1, wherein the first polymer has a carboxylate (COO⁻) or carboxyl group (COOH)

3. The method as claimed in claim 2, wherein the first polymer is alginic acid, alginate, propylene glycol alginate, carboxylmethyl cellulose, polyacrylic acid, or polyacrylate derivatives.

4. The method as claimed in claim 3, wherein the first polymer is sodium alginate.

5. The method as claimed in claim 1, wherein the second polymer is chitosan, starch, dextran, hydroxyl propyl methyl cellulose, or gelatin.

6. The method as claimed in claim 5, wherein the second polymer is chitosan.

7. The method as claimed in claim 1, further comprising, after the emulsion is formed and before separation, adding a cross-linking agent.

8. The method as claimed in claim 1, wherein the second polymer aqueous solution has pH of 0.5 to 6.

9. The method as claimed in claim 1, wherein the second polymer aqueous solution has a weight 1.5 to 20 times the weight of the first polymer aqueous solution.

10. A method for preparing polymeric microsphere encapsulated with a drug, comprising the following steps:
providing an aqueous solution of a first polymer, the first polymer having a functional group capable of forming surface cross-linking;
providing an acidic aqueous solution of a second polymer, wherein the first and second polymer aqueous solutions are miscible;
mixing a drug and the first polymer aqueous solution to form a drug aqueous solution;
mixing the drug aqueous solution and the second polymer aqueous solution and stirring to form an emulsion, such that the first polymer aqueous solution forms a dispersed phase in a continuous phase of the second polymer aqueous solution, wherein the dispersed phase includes a plurality of the first polymeric microsphere encapsulated with the drug, having a microsphere surface composed of a solidification film formed by cross-linking of the functional group; and
separating the first polymeric microsphere out.

11. The method as claimed in claim 10, wherein the first polymer has a carboxylate (COO⁻) or carboxyl group (COOH)

12. The method as claimed in claim 11, wherein the first polymer is alginic acid, alginate, propylene glycol alginate, carboxylmethyl cellulose, polyacrylic acid, or polyacrylate derivatives.

13. The method as claimed in claim 12, wherein the first polymer is sodium alginate.

14. The method as claimed in claim 10, wherein the second polymer is chitosan, starch, dextran, hydroxyl propyl methyl cellulose, or gelatin.

15. The method as claimed in claim 14, wherein the second polymer is chitosan.

16. The method as claimed in claim 10, further comprising, after the emulsion is formed and before separation, adding a cross-linking agent.

17. The method as claimed in claim 10, wherein the second polymer aqueous solution has pH of 0.5 to 6.

18. The method as claimed in claim 10, wherein the second polymer aqueous solution has a weight 1.5 to 20 times the weight of the first polymer aqueous solution.
